(19) United States
(12) Patent Application Publication
(10) Pub. No.: US 2010/0137203 A1
(43) Pub. Date: Jun. 3, 2010

(54) METHOD FOR PREPARING A CELL-DERIVED EXTRACELLULAR MATRIX MEMBRANE

(75) Inventors: Byoung-Hyun Min, Seoul (KR); So Ra Park, Seoul (KR); Byung Hyune Choi, Seoul (KR)

Correspondence Address: MOORE & VAN ALLEN PLLC, P.O. BOX 13706, Research Triangle Park, NC, 27709, US

(73) Assignee: REGENPRIME CO., LTD., Gyeonggi-do (KR)

(21) Appl. No.: 12/442,499

(22) PCT Filed: Apr. 25, 2007
(86) PCT No.: PCT/KR07/02033

§ 371 Date: Feb. 17, 2010

(30) Foreign Application Priority Data
Sep. 21, 2006 (KR) 10-2006-0091777

(51) Int. Cl. A61K 38/16; C12P 19/26; C12N 5/00; A61P 43/00

(52) U.S. Cl. 514/12; 435/84; 435/401

(57) ABSTRACT

The present invention relates to a method for preparing a cell-derived extracellular matrix membrane, more particularly, to a method for preparing a chondrocyte-derived ECM membrane, the method comprising the steps of forming a suitable thickness of ECM membrane by culturing chondrocytes derived from animal cartilage at a high concentration in vitro, and drying it after decellularization process. The cell-derived ECM membrane scaffold according to the present invention is composed of extracellular matrix secreted by chondrocytes so that the membrane has excellent biocompatibility as well as an immune-previlage effect specific to cartilage. Since the membrane also has a suitable compressive strength, it can be used to replace periosteum for cartilage regeneration or artificial collagen membrane and used as dura mater transplant material, a natural ECM membrane for treating skin loss, materials for cell transplantation and a growth factor delivery vehicle.

### TECHNICAL FIELD

The present invention relates to a method for preparing a cell-derived extracellular matrix membrane (ECM membrane), more particularly, to a method for preparing a chondrocyte-derived ECM membrane, which comprises the steps of culturing chondrocytes derived from animal cartilage to form chondrocyte/ECM membrane, removing chondrocytes from the membrane, constructing a suitable thickness of ECM membrane and drying it for sterilization.

### BACKGROUND ART

Articular chondrocytes are specialized mesoderm-derived cells found in only cartilage. Cartilage is an avascular tissue with characteristic physical properties depending on extracellular matrix produced by chondrocytes. Once cartilage is damaged, its self-regeneration is extremely limited to ultimately cause osteoarthritis, which largely affects the quality of patients' lives.

Typical treatments for damaged cartilage include bone marrow stimulation to induce bone marrow-derived stem cells to the damaged site (bone drilling, microfracture, abrasion arthroplasty) and autologous chondrocyte implantation (ACI). Although the bone marrow stimulation is frequently used because of its minimal invasion procedure using an arthroscope in a short period of time, bone marrow-derived blood clots (including stem cells) are not maintained well during the operation so that the regenerated cartilage becomes fibrous cartilage rather than normal cartilage. Therefore, it is difficult to expect successful healing using the bone marrow stimulation. In case of using ECM membrane of the present invention, the possibility of regeneration into a normal cartilage is increased because blood clots derived from bone marrow can be physically maintained.

Although autologous chondrocyte implantation (ACI) is a clinically approved cell transplantation treatment (Brittberg, M. et al., New Eng. J. Med., 331:889, 1994), there is a problem in that the area of cartilage loss should be sutured after collecting the periosteum and the periosteum may be overgrown to cause pain at affected area after the surgery. Moreover, it is troublesome to undergo a surgery process comprising two steps of isolating chondrocytes under arthroscopic operation to culture them for a long time in vitro, and then transplanting a cell suspension into a damaged area. Therefore, in order to solve the problems, it is necessary to improve techniques for surgical operation and periosteum replacement.

The present inventors considered that successful treatment for regenerating hyaline cartilage tissue can be achieved if an ECM membrane, which is a structurally complicated but well-organized compound of various natural proteins, is used as a periosteum replacement, or chondrocytes are attached to ECM membrane to be transplanted.

Previously, allogenic or xenogenic ECM membrane was directly harvested from a living tissue and acellularized to use as membrane-type scaffolds. Representative examples are small intestine submucosa (SIS), urinary bladder submucosa (UBS), human amniotic membrane (HAM) and the like. HAM is useful for cornea regeneration, and SIS is used for the regeneration of urinary tract and dura mater, and vascular reconstruction. And studies on cartilage regeneration using type I, III collagen bilayer membrane are also being conducted.

A chondrocyte-derived ECM scaffold consists basically of glycosaminoglycan (GAG) and collagen, which are main components of the extracellular matrix of cartilage tissue, and includes microelements which are important in chondrocyte metabolism. ECM scaffold provides a natural environment for chondrocyte differentiation and can be applied to the tissue-engineering field as a high quality scaffold.

Recently, it has been reported that use of amniotic membrane as a substrate or basement membrane for cell culture and a method for preparing a cellular therapeutic agent using the same (KR10-2004-7017580). The main component of amniotic membrane is type I collagen and thus there is a shortcoming of low biocompatibility with chondrocytes compared to the ECM membrane. Also, because biodegradation of amniotic membrane cannot be controlled, it may remain in the body even long after transplantation. Also, there is another difficulty of collecting tissues under a donor agreement.

Accordingly, the present inventors have made extensive efforts to develop a membrane-type scaffold prepared in vitro, which has a suitable thickness and compression strength, has no inflammatory response when transplanted, and has excellent biocompatibility enabling it to be applied to clinical practices. As a result, they have prepared an ECM membrane scaffold having a suitable thickness and compressive strength by monolayer-culturing chondrocytes at a high density in vitro to produce a chondrocyte/ECM membrane, removing cells and then drying it naturally, and found that chondrogenic differentiation can be performed for a long period of time when the prepared ECM membrane is transplanted for blood clot protection or periosteum replacement after bone marrow stimulation, thereby completing the present invention.

### SUMMARY OF THE INVENTION

Accordingly, a main object of the present invention is to provide a method for preparing an ECM membrane for tissue engineering by culturing cells at high density in vitro.

Another object of the present invention is to provide a method for preparing a decellularized ECM membrane by removing cells from the ECM membrane.

Another object of the present invention is to provide a cellular therapeutic agent containing the ECM membrane onto which chondrocytes, nerve cells, muscle cells, pancreatic cells, liver cells and stem cells are seeded.

In order to achieve the above objects, the present invention provides a method for preparing a chondrocyte-derived ECM membrane, the method comprising the steps of: (a) isolating chondrocytes from animal-derived cartilage and then culturing them; (b) obtaining a chondrocyte/ECM membrane from the cultured chondrocytes; and (c) obtaining an ECM membrane by removing cells from the chondrocyte/ECM membrane and drying the resultant membrane, and an ECM membrane prepared by the same method.

The present invention also provides a method for preparing a decellularized ECM membrane, the method comprises removing cells from the ECM membrane, and a decellularized ECM membrane prepared by the same method.

The present invention also provides a method for preparing a decellularized ECM membrane, the method comprising the steps of: (a) producing a chondrocyte/ECM membrane by isolating chondrocytes from animal-derived cartilage to culture; (b) obtaining a decellularized ECM membrane construct by removing chondrocytes from the produced chondrocyte/ECM membrane; and (c) obtaining a decellularized ECM membrane by drying the obtained decellularized ECM membrane construct, and a decellularized ECM membrane prepared by the same method.

The present invention also provides a method for preparing an intensive ECM membrane, the method comprises increasing thickness of the membrane by multi-layering at least one selected from the group consisting of the ECM membrane and the decellularized ECM membrane.

The present invention also provides a method for preparing various shapes of ECM membranes, the method comprises processing at least one selected from the group consisting of the ECM membrane and the decellularized ECM membrane.

The present invention also provides a method for preparing a cell-attached ECM membrane, the method comprises drying at least one selected from the group consisting of the ECM membrane and the decellularized ECM membrane, in a cell culture dish, and then seeding cells onto the surface of said ECM membrane or said decellularized ECM membrane, and a cellular therapeutic agent containing a cell-seeded ECM membrane prepared by the same method.

The present invention also provides a method for preparing a growth factor-attached ECM membrane, the method comprises attaching growth factors to at least one selected from the group consisting of the ECM membrane and the decellularized ECM membrane.

The present invention also provides a method for preparing an intensive ECM membrane for the controlled release of growth factors, the method comprises multi-layering the growth factor-attached ECM membrane prepared by the above-mentioned method.

The present invention also provides a drug delivery carrier for the controlled release of growth factors, which comprise the growth factor-attached ECM membrane prepared by the above-mentioned method and a drug delivery carrier for the controlled release of growth factors, which comprises the intensive ECM membrane for the controlled release of growth factors.

Another features and embodiments of the present invention will be more clarified from the following detailed description and the appended claims.

### BRIEF DESCRIPTION OF DRAWINGS

FIG. 1 is a photograph of the ECM membrane prepared by the present invention.

FIG. 2 is SEM (scanning electron microscope) images of the surface (A, 50×) and the cross section (B, 1500×) of the ECM membrane according to the present invention.

FIG. 3 is tissue images obtained after the ECM membrane according to the present invention is stained with hematoxylin/eosin (original magnification, A: 200×, B: 400×).

FIG. 4 is the result of comparing secondary chemical structure by infrared spectrum analysis of the ECM membrane according to the present invention and natural pig cartilage tissue.

FIG. 5 is the result of examining cell proliferation capacity by MTT assay after culturing rabbit chondrocytes on an ECM membrane (B) according to the present invention and a commercial culture dish (A).

FIG. 6 is the result of examining the histological change and proteoglycan expression by hematoxylin/eosin staining (A) and Safranin staining (B) at 7 and 14 days after culturing rabbit chondrocytes on the ECM membrane according to the present invention.

FIG. 7 is the result of morphological analysis (A), SEM image (B), and histological analysis (H&E staining, C) after removing cells by decellularization process of the ECM membrane according to the present invention.

FIG. 8 is the result of DAPI staining (A) and quantitative analysis (B) of the amount of remaining DNA before and after decellularizing the ECM membrane according to the present invention.

FIG. 9 is the result of examining collagen content (A), proteoglycan content (B), and total protein content (C) in samples before and after decellularizing the ECM membrane according to the present invention.

FIG. 10 is the result of examining secondary chemical structure of samples by infrared spectrum analysis before and after decellularizing the ECM membrane according to the present invention.

FIG. 11 is the result of measuring the tensile strength and expansion rate of the decellularized ECM membrane according to the present invention after it becomes two-layered or three-layered to enhance its thickness.

### DETAILED DESCRIPTION OF THE INVENTION, AND PREFERRED EMBODIMENTS

In one aspect, the present invention relates to a method for preparing an ECM membrane for tissue engineering through a high density culture and the ECM membrane prepared by the same method. Specifically, the present invention relates to a method for preparing a chondrocyte-derived ECM membrane, the method comprising the steps of: (a) isolating chondrocytes from animal-derived cartilage and then culturing them; (b) obtaining a chondrocyte/ECM membrane from the cultured chondrocytes; and (c) obtaining an ECM membrane by removing cells from the chondrocyte/ECM membrane and drying the resultant membrane, and an ECM membrane prepared by the same method.

In the present invention, the above mentioned method preferably comprises an additional step (d) obtaining a thick ECM membrane with a high compressive strength by reseeding chondrocytes onto the obtained ECM membrane to re-culture.

In one embodiment of the present invention, chondrocytes were used to prepare an ECM membrane having a compressive strength suitable for transplantation by controlling thickness, which is biocompatible, cell-friendly and immunocompatible. The ECM membrane can not only replace periosteum used for cartilage regeneration or artificial collagen membrane but also be used as a scaffold for cell transplantation due to its capability of promoting chondrocyte proliferation and performing chondrogenic differentiation for a long period of time, and as a transplant material for treating dura mater loss, skin loss and nervous tissue damage.

In the present invention, the animal is preferably a pig or a human, and bioactive factors can preferably additionally be added to the culture.

In the present invention, the culture broth is preferably treated with ultrasonic waves or physical pressure is applied thereto in the culture step, and the drying of the step (c) is preferably performed by repeating the procedure of freezing and thawing the chondrocyte/ECM membrane construct at −15˜−25° C., 3˜5 times to naturally dry or freeze-dry.

In one embodiment of preparing method of the ECM membrane according to the present invention, chondrocytes isolated from pig cartilage are monolayer-cultured at a high density for 3˜4 weeks, and then the chondrocyte/ECM membrane is dried at 4° C. to prepare the ECM membrane containing the cartilage-specific extracellular matrix.

The ECM membrane prepared according to one embodiment of the present invention is a 10˜20 μm thickness of biomembrane, which has collagen and proteoglycan as main components, and its compressive strength is 25N/mm² and its expansion rate is 10%.

When chondrocytes are cultured on the ECM membrane according to the present invention, cell proliferation capacity is equal to that when cultured in a conventional animal cell culture dish and proteins, specific to chondrocytes such as proteoglycan, are well expressed.

In the present invention, the step of culturing chondrocytes are preferably performed by culturing them with more than one selected from the group consisting of myoblasts, myocytes, cardiomyocytes, neurons, fibroblasts, fibrocytes, osteoblasts and stem cells.

In another aspect, the present invention relates to a method for preparing a decellularized ECM membrane, the method comprises removing cells from the ECM membrane, and a decellularized ECM membrane prepared by the same method.

In the present invention, the decellularization can preferably be performed by treating the ECM membrane with more than one selected from the group consisting of ionic detergents, nonionic detergents, denaturants, hypotonic solution, DNase, RNase and ultrasonic waves, and the decellularization can preferably be performed at a temperature range of 0˜50° C.

In still another aspect, the present invention relates to a method for preparing a decellularized ECM membrane, the method comprising the steps of: (a) producing a chondrocyte/ECM membrane by isolating chondrocytes from animal-derived cartilage to culture; (b) obtaining a decellularized ECM membrane construct by removing chondrocytes from the produced chondrocyte/ECM membrane; and (c) obtaining a decellularized ECM membrane by drying the obtained decellularized ECM membrane construct, and a decellularized ECM membrane prepared by the same method.

In the present invention, chondrocyte removal of the (b) step is preferably performed by treating the chondrocyte/ECM membrane with more than one selected from the group consisting of ionic detergents, nonionic detergents, denaturants, hypotonic solution, DNase, RNase and ultrasonic waves, and the decellularization is preferably performed at a temperature range of 0˜50° C.

By multi-layering the ECM membrane according to the present invention or the decellularized ECM membrane, an ECM membrane, whose thickness has increased, can be prepared. Therefore, the present invention, in still another aspect, relates to a method for preparing an intensive ECM membrane, the method comprises increasing membrane thickness by multi-layering the ECM membrane or the decellularized ECM membrane.

Moreover, when the ECM membrane or the decellularized ECM membrane according to the present invention is processed, various shapes of ECM membranes can be obtained. Therefore, the present invention, in still another aspect, relates to a method for preparing various shapes of ECM membranes, the method comprises processing the ECM membrane or the decellularized ECM membrane.

Meanwhile, since the ECM membrane or the decellularized membrane according to the present invention has high cell proliferation rate and excellent phenotype maintenance capacity in vitro, a cellular therapeutic agent that can be applied to clinical practices, can be prepared by attaching cells to be treated to the surface thereof. Thus, the present invention, in still another aspect, relates to a method for preparing a cell-attached ECM membrane, the method comprises drying the ECM membrane or the decellularized ECM membrane in a cell culture dish, and then seeding the cells onto the surface of said ECM membrane or said decellularized ECM membrane, and relates a cellular therapeutic agent containing a cell-seeded ECM membrane prepared by the same method.

In the present invention, the cells are selected from the group consisting of chondrocytes, skin cells, neurons, muscle cells, pancreatic cells, liver cells and stem cells.

In the present invention, the cellular therapeutic agent is preferably for treating cerebral dura mater or regenerating thereof, regenerating skin and cartilage, stanching the bleeding in internal organs, and regenerating tissues of internal organs.

Also, by attaching growth factors necessary for cell proliferation to the ECM membrane or the decellularized ECM membrane, the membrane can be used as a drug delivery carrier to release growth factors in vivo. Therefore, the present invention, in still another aspect, relates to a method for preparing a growth factor-attached ECM membrane, the method comprises attaching growth factors to the ECM membrane or the decellularized ECM membrane and a drug delivery carrier for the controlled release of growth factor, which comprises the ECM membrane having the growth factors attached.

In one aspect of the present invention, the growth factors attached to the ECM membrane include insulin-like growth factor (IGF), basic fibroblast growth factor (bFGF), acidic fibroblast growth factor (aFGF), transforming growth factor-alpha (TGF-α), transforming growth factor-beta (TGF-β) bone morphogenetic protein (BMP), platelet-derived growth factor (PDGF), keratinocyte growth factor (KGF), epidermal growth factor (EGF), vascular endothelial growth factor (VEGF), Erythropoietin (EPO), granulocyte macrophage colony stimulating factor (GM-CSF), granulocyte colony stimulating factor (G-CSF), nerve growth factor (NGF), heparin binding epidermal growth factor (heparin binding EGF) and the like, but it is not limited thereto.

In one embodiment of the present invention, an intensive ECM membrane with a high compressive strength was produced by applying physical pressure to the ECM membrane to make multilayers. The intensive ECM membrane is prepared by multi-layering the growth factor-attached ECM membrane, and thus it can be used as a slow-releasing drug delivery carrier which gradually releases growth factors. Thus, the present invention, in still another aspect, relates to a method for preparing an intensive ECM membrane for the controlled release of growth factors, the method comprises multi-layering the growth factor-attached ECM membrane, and a drug delivery carrier for the controlled release of growth factors, which contains the intensive ECM membrane for the controlled release of growth factors prepared by above method.

### EXAMPLES

Hereinafter, the present invention will be described in more detail by examples. It will be obvious to a person skilled in the art, however, that these examples are for illustrative purpose only and are not construed to limit the scope of the present invention.

Particularly, the following examples describe a method for preparing the ECM membrane using pig articular cartilage according to the method of the present invention, however, it will be obvious to a person skilled in the art that an ECM membrane is prepared using cartilage from other animals.

Also, the following examples exemplify a method for preparing the ECM membrane and the decellularized ECM membrane according to the embodiments of the present invention, however, it will be obvious to a person skilled in the art that a thick ECM membrane having a high compressive strength is prepared by seeding chondrocytes onto the chondrocyte/ECM membrane obtained according to the present embodiment to culture several times.

Also, it will be obvious to a person skilled in the art that an ECM membrane can be prepared using other somatic cells which produce extracellular matrix, except cartilage, according to the method of the present invention.

### Example 1

### Isolation of Pig Chondrocytes

2-week-old pigs without cholera, other viruses and contagious diseases were excessively anesthetized to kill according to animal ethics policy. In a sterile environment, cartilages were harvested from the stifle joint. The harvested cartilages were sliced into small pieces on a clean work table and treated with 0.1% collagenase for 12 hours. After filtering cells with a 0.4 μm filter, chondrocytes were isolated by centrifugation.

### Example 2

### Preparing of a Chondrocyte-Derived ECM Membrane

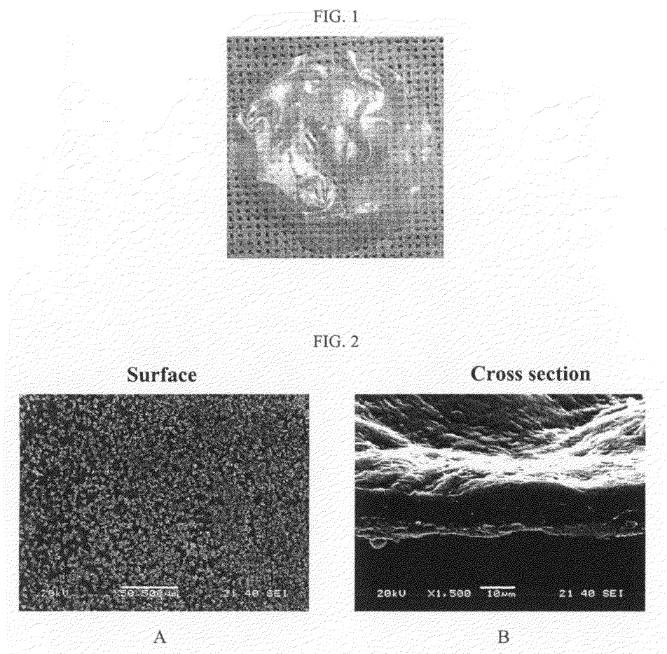

The chondrocytes isolated in Example 1 were seeded onto a 6-well culture dish at a concentration of 0.7×10⁵ cells/cm² and then cultured in a culture medium (DMEM+20% FBS+1% penicillin-streptomycin+5 μg/mL ascorbic acid) for 3 weeks. The culture medium was replaced every three days. After 3 weeks, the ECM film was washed with PBS three times and separated from the 6-well culture dish. The prepared ECM membrane exhibited a semi-transparent thin membrane type (FIG. 1)

### Example 3

### Analysis of Physicochemical Properties of an ECM Membrane

### 3-1: Microstructure Analysis of an ECM Membrane

The microstructure of an ECM membrane was analyzed by scanning electron microscope (SEM). The ECM membrane prepared in Example 2 was fixed with 2.5% glutaraldehyde for 1 hour and washed with phosphate buffer solution. After dehydrating specimens with ethanol to dry, their surface and cross section were observed by an electronic microscope (JEOL, JSM-6380, 20KV, Japan). As a result, tough surface was observed due to structural bodies which seem to be cells and the cross section displayed about 10˜20 μm thickness.

### 3-2: Histological Observation of an ECM Membrane

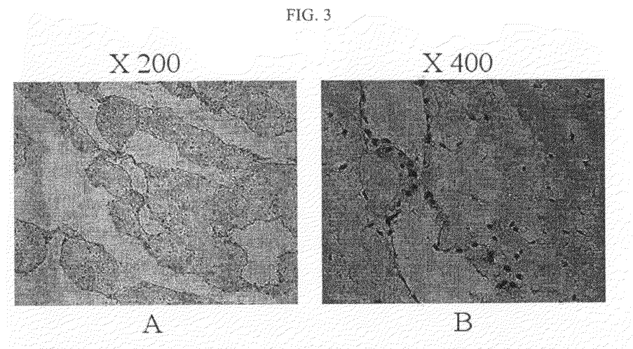

The ECM membrane prepared in Example 2 was fixed with 4% formalin solution for 24 hours, then embedded in paraffin and sectioned. The cross sections were stained with hematoxylin and eosin (H&E). The observation presented that cells having a nucleus were scattered inside of structural bodies which appear to be ECM membranes (FIG. 3).

### 3-3: Measurement of Compressive Strength of an ECM Membrane

Compressive strength of an ECM membrane was measured using a Universal Testing Machine (H5K-T, HTE, Salfords, England). The ECM membrane prepared in Example 2 was cut into uniform rectangular (30×5 mm) and vertically fixed to a 50N load cell. The strength of cut specimens was measured by performing axial pull-out testing at a crosshead speed of 10 mm/min. Also, after measuring several specimens to obtain an average value, the compressive strength per unit area was calculated, thereby obtaining the compressive strength of about 25N/mm² and the expansion rate of 10%.

### 3-4: Comparison of Secondary Structures of an ECM Membrane and Cartilage Tissue

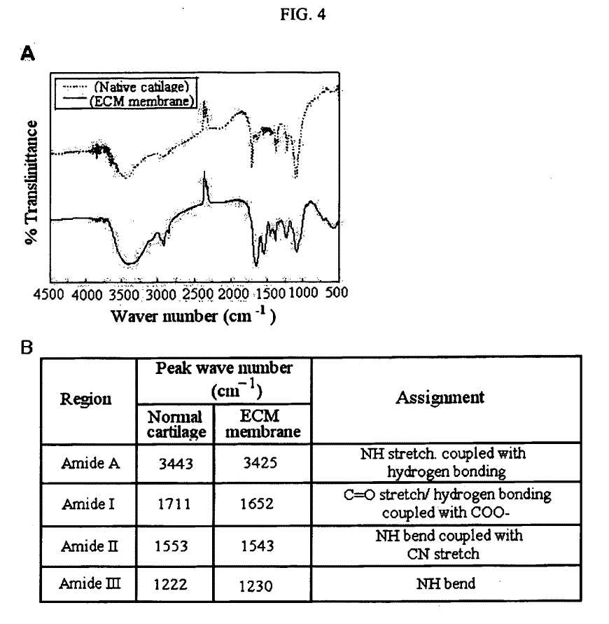

Infrared spectrum analysis was utilized to compare secondary structures between the ECM membrane prepared in Example 2 and pig cartilage tissue. The FT-IR analyzer (Bomem, MB104) with 8 cm⁻¹ resolution was used to analyze amide which is the main component of a protein, and the result revealed that the prepared ECM membrane and natural pig cartilage tissue have similar secondary chemical structure (FIG. 4).

### Example 4

### Chondrocyte Culture Using an ECM Membrane

Using the same method as described in Example 1, chondrocytes were isolated from cartilage tissue of New Zealand white rabbits (about 3˜4 kg). The isolated rabbit chondrocytes were seeded onto the ECM membrane prepared in Example 2 at a concentration of 1×10⁵ cells/30 mm², and then cell proliferation capacity, histological change and proteoglycan expression were examined.

4-1: Examination of Cell Proliferation Capacity with the Passage of Time

Rabbit chondrocytes were cultured in a traditional culture dish for animal cells (control group, 6-well culture plate, BD falcon, USA) and on the ECM membrane, respectively, and cell proliferation capacity was examined by MTT assay (Roche, Germany) at 1, 2, 4, 6, 8, 12, 14 days after cultivation. As a result, cells of the ECM membrane and the commercial culture dish reached a plateau at 5˜6 days as well as the OD value of both groups was also similarly 0.4 (FIG. 5). From the results, it was found that the ECM membrane provides an appropriate environment for chondrocyte proliferation, like the conventional culture dish.

### 4-2: Examination of Histological Change and Proteoglycan Expression

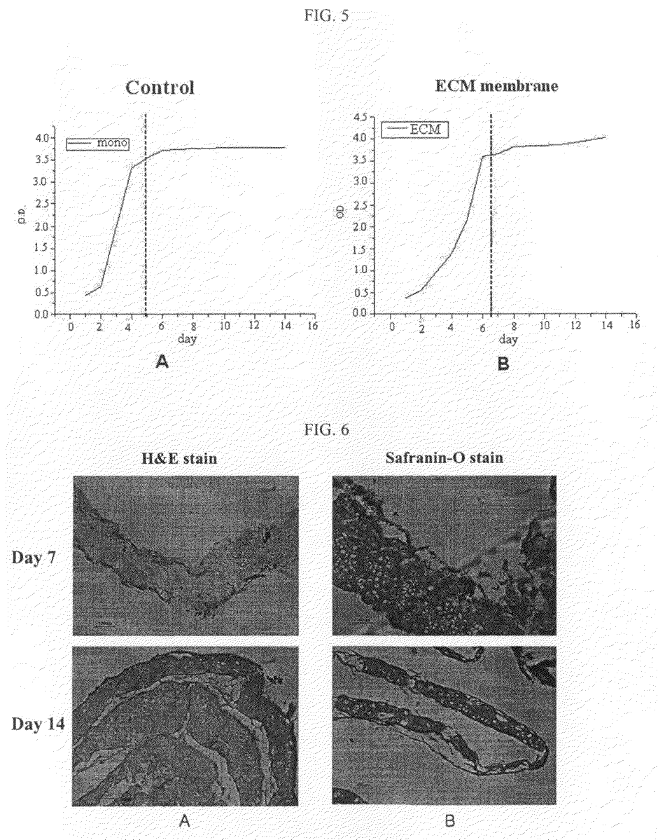

After culturing chondrocytes on the ECM membrane as described above, cells were collected at 7, 14 days to make tissue sections using the same method as Example 3-2. The sections were stained with hematoxylin and eosin (H&E), and as a result, it was confirmed that thick cell layers were formed on the surface of the ECM membrane with time (FIG. 6A). Also, Safranin O staining which is specific to proteoglycan exhibited that proteoglycans were remarkably expressed all over the specimens (FIG. 6B).

### Example 5

### Preparing of an Decellularized ECM Membrane and its Characteristic Analysis

### 5-1: Decellularization of an ECM Membrane

In order to remove chondrocytes exist in the ECM membrane and obtain pure ECM membrane, a decellularization process was performed as follows. The ECM membrane prepared in Example 2 was put into in 0.1% SDS solution and agitated at 37° C. at 150 rpm for 24 hours. Next, the membrane was treated by the following procedure: 1 minute in an ultrasonic washer, 30 minutes in 0.05% trypsin-EDTA solution, 1 minute in an ultrasonic washer, 24 hours in 0.07 mg/mL DNase solution and 1 minute in an ultrasonic washer. After that the membrane was washed with PBS at least five times. The ECM membrane, which has undergone decellularization process, was dried in a hood for 12 hour and stored in an electronic desiccator.

### 5-2: Morphological and Histological Analysis of a Decellularized ECM Membrane

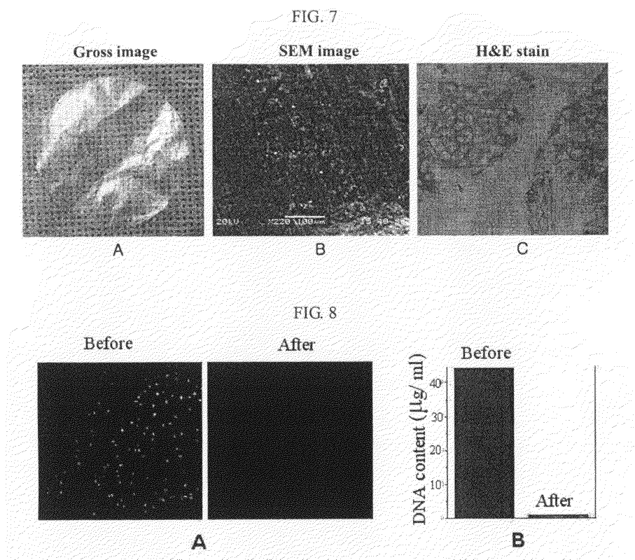

Although the decellularized ECM membrane became thinner (⅓) than the ECM membrane before decellularization, there was no significant differences between them in overall morphology, color and tactile sense (FIG. 7A). The result from microstructure analysis of the surface using a scanning electron microscope by the same method as Example 3-1 exhibited that white cellular bodies, which were detected in specimens (FIG. 2A) before decellularization, were not observed as well as the decellularized membrane showed an overall smooth shape (FIG. 7B). Also, the histological observation using H&E staining as described in Example 3-2 revealed that small and dense nuclear bodies, which appeared in specimens before decellularization, were not observed (FIG. 7C).

### 5-3: DNA Content Analysis of a Decellularized ECM Membrane

Using the same method as Example 3-2, sections from the ECM membrane before decellularization and the ECM membrane after decellularization were prepared to stain with 200 ng/ml of DAPI [2-(4-Amidinophenyl)-6-indolecarbamidine Dihydro chloride] solution, and then observed by a fluorescent microscope. As a result, DNA contents in nuclear bodies were distinctly observed in specimens before decellularization, but fluorescence-labeled DNA contents were not detected at all in specimens after decellularization (FIG. 8A). The effect of DNA removal in the decellularized ECM membrane was also manifested in the result of quantitative analysis of DNA contents using Hoechst 332582 staining material (FIG. 8B).

### 5-4: Component Analysis of a Decellularized ECM Membrane

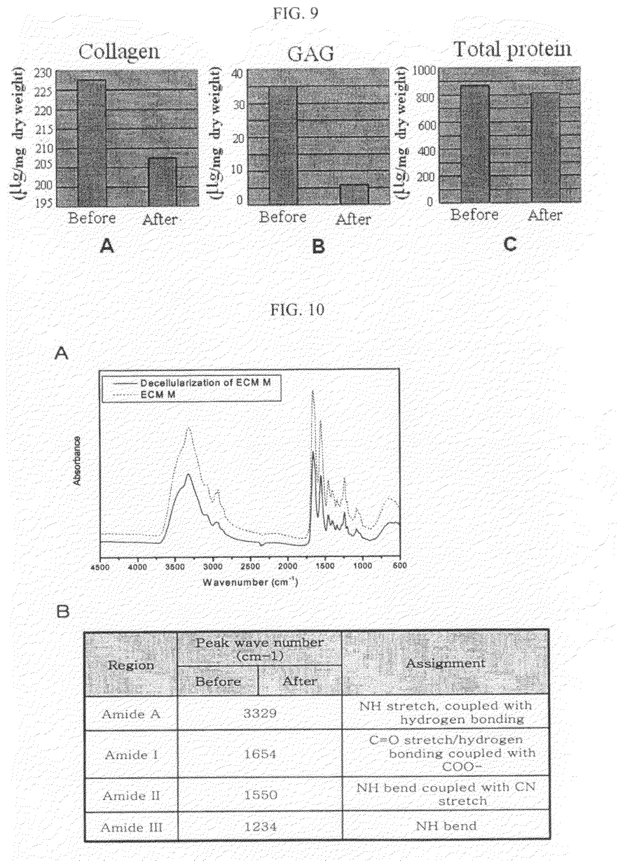

The contents of collagen and proteoglycan, which are main ECM components of cartilage tissue, as well as the contents of total proteins were compared and analyzed using specimens ECM membranes before and after decellularization, respectively. As a result, it was found that the decellularized ECM membrane showed a remarkable decrease in the contents of collagen and proteoglycan per unit weight, but no difference in the contents of total proteins when compared to the ECM membrane before decellularization (FIG. 9).

Collagen contents were measured as follows. The dried ECM membrane specimens were placed in 1 mL of 1N choleric acid solution and left to stand at 60° C. for 24 hours, and then hydrolyzed by high-pressure sterilizing treatment in 2N Sodium hydroxide solution (NaOH) at 120° C. for 20 minutes. After 450 μL of chloramines-T reagent was added to the specimens and left to stand at an ambient temperature for 25 minutes, 500 μL of Endlich's reagent for color development was added and left to stand at 60° C. for 20 minutes, followed by measuring their absorbance at 550 nm wavelength. The final concentration of collagen was calculated using a hydroxyproline standard curve.

Proteoglycan contents were measured as follows. The dried ECM membrane was dissolved in 1 mL of papain solution at 60° C. for 24 hours and then was centrifuged at 10,000 rpm for 3 minutes to separate the supernatant, which was used as a specimen. 50 μL of the supernatant was dispensed into each well of a 96-well culture plate. 200 μL of DMB color fixation solution (obtained by adding 16 mg of DMB, 5 mL of 95% ethanol, 3 mL of formic acid and 25.6 mL of 1M sodium hydroxide in 1 L of ultra-pure water; pH 3.5) was added to each well and allowed it to react at an ambient temperature for 30 minutes, thereby measuring absorbance at 530 nm wavelength. A standard curve created using chondroitin sulphate C was used to produce the concentration of specimens.

The contents of total proteins of specimens were measured as follows. Using the same measurement as that of proteoglycan contents, the ECM membrane extract eluted from papain solution was diluted at 1/10, 1/20 and 1/40, respectively, and then 20 μL of each dilution was dispensed into a 96-well culture plate. Then, BCA reaction solution (Pierce, USA) was added to each well and allowed it to react at an ambient temperature for 30 minutes, thereby measuring their absorbance at 562 nm wavelength. A standard curve created using bovine serum albumin (BSA, 2 mg/mL) was used to produce the final concentration.

### 5-5: Secondary Structure Analysis of a Decellularized ECM Membrane

Using the same method as Example 3-4, the secondary structure of the ECM membrane before and after decellularization was analyzed by FT-IR analysis. As a result, it was confirmed that overall absorbance was similar in specimens of two groups (before- and after-decellularization). Analysis of amide also exhibited that similar structures were found in two groups (FIG. 10).

### Example 6

### Preparing of an Overlapped Intensive ECM Membrane

Using the same method as Example 5, the decellularized ECM membrane was multi-layered two layers or three layers by a compression method to prepare the ECM membrane whose thickness and strength were intensified. The intensified ECM membrane multilayer had a similar shape (which is not largely different from the membrane before decellularization), and its thickness was 3.3 μm (1 layer), 6.6 μm (2 layers) and 10 μm (3 layers), respectively. In case of the 3-layered specimen, it showed the similar thickness to the ECM membrane before decellularization.

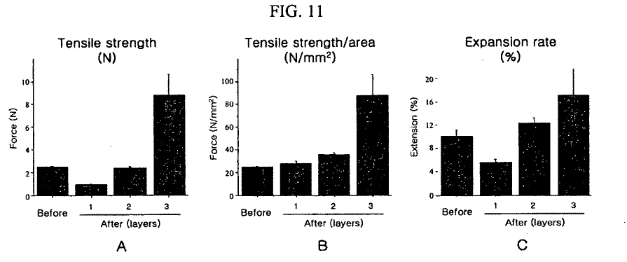

The compressive strength and expansion rate of this membrane were measured by the same method as Example 3-3. As a result, the compressive strength of the decellularized ECM membrane was reduced when compared to the ECM membrane before decellularization, but the compressive strength per unit area has no significant difference (FIG. 11). Also, when the decellularized ECM membrane was multi-layered (2 layers and 3 layers), both the total compressive strength and the compressive strength per unit area increased. In case of the 3-layered specimen whose thickness is similar to that of the specimen before decellularization, about 3.5-fold compressive strength was shown. The expansion rate also decreased a little after decellularization, but increased with intensification by multi-layering.

### INDUSTRIAL APPLICABILITY

As described in detail above, the present invention has an effect to provide a method for preparing a membrane-type scaffold containing extracellular matrix secreted by chondrocytes and a cell-derived ECM membrane prepared by the same method. The cell-derived ECM membrane scaffold according to the present invention is composed of extracellular matrix secreted by chondrocytes so that the membrane has excellent biocompatibility as well as the effect of immune-previlage specific to cartilage. Since the membrane also has a compressive strength suitable for transplantation, it can be used to replace periosteum for cartilage regeneration or artificial collagen membrane and used as dura mater transplant material, a natural ECM membrane for treating skin loss, materials for cell transplantation and a growth factor delivery vehicle.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A method for preparing a chondrocyte-derived ECM membrane, the method comprising the steps of:
(a) isolating chondrocytes from animal-derived cartilage and then culturing them;
(b) obtaining a chondrocyte/ECM membrane from the cultured chondrocytes; and
(c) obtaining an ECM membrane by removing cells from the chondrocyte/ECM membrane and drying the resultant membrane.

2. The method for preparing a chondrocyte-derived ECM membrane according to claim 1, which additionally comprises a step (d) obtaining a thick ECM membrane with a high compressive strength by reseeding chondrocytes onto the obtained ECM membrane to re-culture.

3. The method for preparing a chondrocyte-derived ECM membrane according to claim 1, wherein the animal is a pig.

4. The method for preparing a chondrocyte-derived ECM membrane according to claim 1, wherein the animal is a human.

5. The method for preparing a chondrocyte-derived ECM membrane according to claims 1 or 2, wherein bioactive factors are additionally added to the culture step.

6. The method for preparing a chondrocyte-derived ECM membrane according to claim 5, wherein the bioactive factors are more than one selected from the group consisting of insulin-like growth factor (IGF), basic fibroblast growth factor (bFGF), acidic fibroblast growth factor (aFGF), transforming growth factor-alpha (TGF-α), transforming growth factor-beta (TGF-β), bone morphogenetic protein (BMP), platelet-derived growth factor (PDGF), keratinocyte growth factor (KGF), epidermal growth factor (EGF), vascular endothelial growth factor (VEGF), Erythropoietin (EPO), granulocyte macrophage colony stimulating factor (GM-CSF), granulocyte colony stimulating factor (G-CSF), nerve growth factor (NGF), heparin binding epidermal growth factor (heparin binding EGF), interferons, tissue activating peptides, interleukin-1 (IL-1), interleukin-2 (IL-2) interleukin-6 (IL-6) and interleukin-8 (IL-8).

7. The method for preparing a chondrocyte-derived ECM membrane according to claims 1 or 2, wherein the culture broth is treated with ultrasonic waves or physical pressure is applied thereto in the culture step.

8. The method for preparing a chondrocyte-derived ECM membrane according to claim 1, wherein the drying in step (c) is performed by repeating the procedure of freezing and thawing the chondrocyte/ECM membrane construct at −15˜−25° C., 3˜5 times to naturally dry or freeze-dry.

9. The method for preparing a chondrocyte-derived ECM membrane according to claims 1 or 2, wherein the step of culturing chondrocytes is performed by culturing them with more than one selected from the group consisting of myoblasts, myocytes, cardiomyocytes, neurons, fibroblasts, fibrocytes, osteoblasts and stem cells.

10. An ECM membrane prepared by the method of claims 1 or 2.

11. A method for preparing a decellularized ECM membrane, which comprises removing cells from the ECM membrane of claim 10.

12. The method for preparing a decellularized ECM membrane according to claim 11, wherein the decellularization is performed by treating the ECM membrane with more than one selected from the group consisting of ionic detergents, nonionic detergents, denaturants, hypotonic solution, DNase, RNase and ultrasonic waves.

13. A decellularized ECM membrane prepared by the method of claim 11.

14. A method for preparing a decellularized ECM membrane, the method comprising the steps of:
(a) producing a chondrocyte/ECM membrane by isolating chondrocytes from animal-derived cartilage to culture;
(b) obtaining a decellularized ECM membrane construct by removing chondrocytes from the produced chondrocyte/ECM membrane; and
(c) obtaining a decellularized ECM membrane by removing cells from the decellularized ECM membrane and drying the resultant membrane.

15. The method for preparing a decellularized ECM membrane according to claim 14, wherein the chondrocyte removal of the step (b) is performed by treating the chondrocyte/ECM membrane with more than one selected from the group consisting of ionic detergents, nonionic detergents, denaturants, hypotonic solution, DNase, RNase and ultrasonic waves.

16. A decellularized ECM membrane prepared by the method of claim 14.

17. A method for preparing an intensive ECM membrane, which comprises increasing thickness of the membrane by multi-layering at least one selected from the group consisting of the ECM membrane of claim 10, the decellularized ECM membrane of claims 13 and 16.

18. A method for preparing various shapes of the ECM membranes, which comprises processing at least one selected from the group consisting of the ECM membrane of claim 10, the decellularized ECM membrane of claims 13 and 16.

19. A method for preparing a cell-attached ECM membrane, the method comprising the steps of (a) drying at least one selected from the group consisting of the ECM membrane of claim 10, the decellularized ECM membrane of claims 13 and 16, in a cell culture dish, (b) seeding the cells onto the surface of said ECM membrane or said decellularized ECM membrane.

20. The method according to claim 19, wherein the cells are selected from the group consisting of chondrocytes, skin cells, nerve cells, muscle cells, pancreatic cells, liver cells and stem cells.

21. A cellular therapeutic agent containing a cell-seeded ECM membrane prepared by the method of claim 19.

22. The cellular therapeutic agent according to claim 19, wherein it is for treating cerebral dura mater or regenerating thereof, regenerating skin and cartilage, stanching the bleeding in internal organs, and regenerating tissues of internal organs.

23. A method for preparing a growth factor-attached ECM membrane, which comprises attaching growth factors to at least one selected from the group consisting of the ECM membrane of claim 10, the decellularized ECM membrane of claims 13 and 16.

24. A method for preparing an intensive ECM membrane for the controlled release of growth factors, which comprises multi-layering the growth factor-attached ECM membrane prepared by the method of claim 23.

25. A drug delivery carrier for the controlled release of growth factors, which contains the growth factor-attached ECM membrane prepared by the method of claim 23.

26. A drug delivery carrier for the controlled release of growth factors, which contains the intensive ECM membrane for the controlled release of growth factors of claim 24.
